(19) United States
(12) Patent Application Publication
(10) Pub. No.: US 2023/0142701 A1
(43) Pub. Date: May 11, 2023

(54) RESPIRATOR MASK CLEANER

(71) Applicant: Cleanbox Technology, Inc., Nashville, TN (US)

(72) Inventor: DAVID ALLEN GEORGESON, Vista, CA (US)

(21) Appl. No.: 17/918,240

(22) PCT Filed: Apr. 8, 2021
(86) PCT No.: PCT/US21/26483

§ 371 Date: Oct. 11, 2022

Related U.S. Application Data
(60) Provisional application No. 63/007,319, filed on Apr. 8, 2020.

(51) Int. Cl. A61L 2/10

(52) U.S. Cl. CPC A61L 2/10; A61L 2202/122; A61L 2202/11

(57) ABSTRACT

The present disclosure provides cleaning apparatus for respirator masks (e.g., N95 respirator masks) and methods of using same.

### PRIORITY CLAIM

This application is a National Stage entry of International PCT Patent Application Serial No. PCT/US2021/026483, filed on Apr. 8, 2021, which claims priority to U.S. Provisional Patent Application Ser. No. 63/007,319, filed on Apr. 8, 2020, the entire contents of each of which are incorporated herein by reference and relied upon.

### FIELD OF DISCLOSURE

The present disclosure provides cleaning apparatus for respirator masks (e.g., N95 respirator masks) and methods of using same.

### BACKGROUND

Respirator masks (e.g., N95 respirator masks) are difficult to thoroughly clean between uses, and can therefore represent a dangerous safe harbor for pathogens. Cleaning methods involving liquid cleansers suffer from many drawbacks, including unpleasant residues, odors, and even physical damage to the structural and barrier components of the respirator mask.

There remains a need for improved methods for cleaning reusable respirator masks, such as N95 respirator masks, of pathogens between uses. The present disclosure describes systems and methods that meet those needs.

### SUMMARY

The present disclosure provides cleaning apparatus for respirator masks (e.g., N95 respirator masks) and methods of using same.

In some embodiments, the present disclosure provides a respirator cleaning apparatus comprising a housing; a superior lamp; an inferior lamp; and a respirator support disposed between the superior lamp and the inferior lamp.

In some embodiments, the present disclosure provides a method of cleaning a respirator mask, the method comprising: providing a respirator cleaning device of any one preceding claim; inserting the respirator mask into the housing; illuminating the respirator mask with the superior lamp and with the inferior lamp for a period of time sufficient to incapacitate substantially all or all pathogens associated with the respirator mask; and thereafter removing the respirator mask from the housing.

These and other embodiments are described in greater detail herein.

### BRIEF DESCRIPTION OF THE DRAWINGS

The detailed description of some embodiments of the present disclosure is made below with reference to the accompanying figures, wherein like numerals represent corresponding parts of the figures.

FIG. 1A shows a perspective view of a respirator cleaning apparatus consistent with one embodiment of the present disclosure.

FIG. 1B shows a perspective view of the respirator cleaning apparatus of FIG. 1A with the door open and the respirator support partially withdrawn from the chamber.

FIG. 2 shows a perspective view of the respirator cleaning apparatus of FIG. 1B with respirator masks affixed temporarily to the respirator support.

FIG. 3 shows a perspective view of the superior lamp tray and the inferior lamp display of the respirator cleaning apparatus of FIG. 1A.

FIG. 4 shows a perspective view of the respirator cleaning apparatus of FIG. 1A with the respirator support, the superior lamp tray, and the inferior lamp tray removed.

FIG. 5 shows a perspective view of the respirator cleaning apparatus of FIG. 1A.

FIG. 6 shows a perspective view of a respirator support consistent with one embodiment of the present disclosure.

FIG. 7 shows a perspective view of the respirator support of FIG. 6 with respirator masks affixed temporarily thereto.

FIG. 8A shows a side view of superior lamps illuminating a respirator mask consistent with one embodiment of the present disclosure.

FIG. 8B shows a top perspective view of the superior lamps of FIG. 8A illuminating a respirator mask.

FIG. 8C shows a side view of inferior lamps illuminating a respirator mask consistent with one embodiment of the present disclosure.

FIG. 8D shows a bottom perspective view of the inferior lamps of FIG. 8C illuminating a respirator mask.

FIG. 9A shows a side perspective view of superior lamps illuminating a respirator mask consistent with one embodiment of the present disclosure.

FIG. 9B shows a side perspective view of inferior lamps illuminating a respirator mask consistent with one embodiment of the present disclosure.

### DETAILED DESCRIPTION

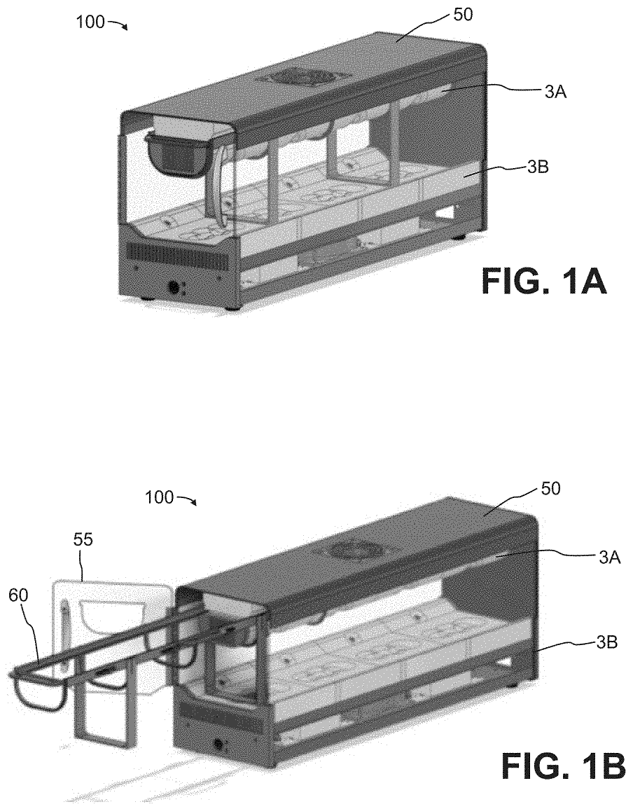

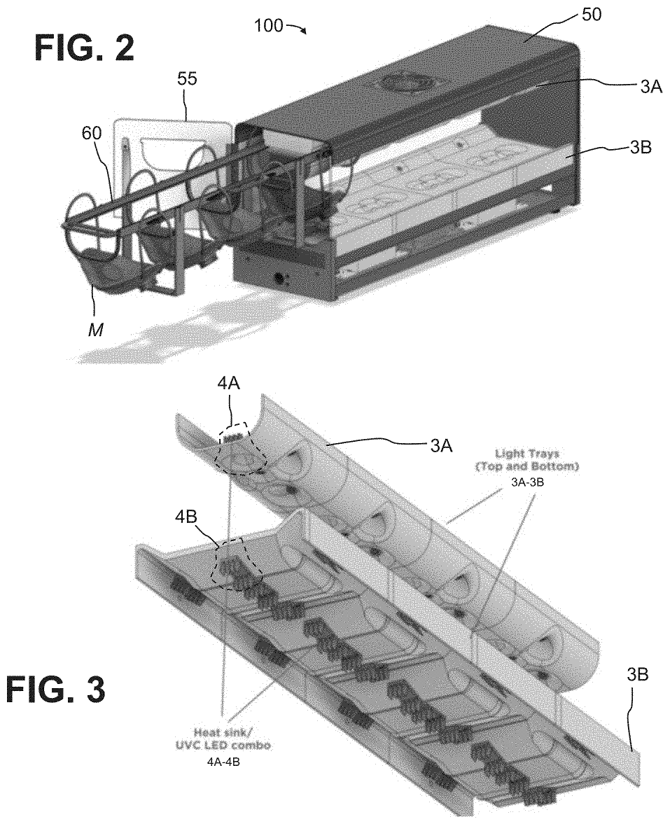

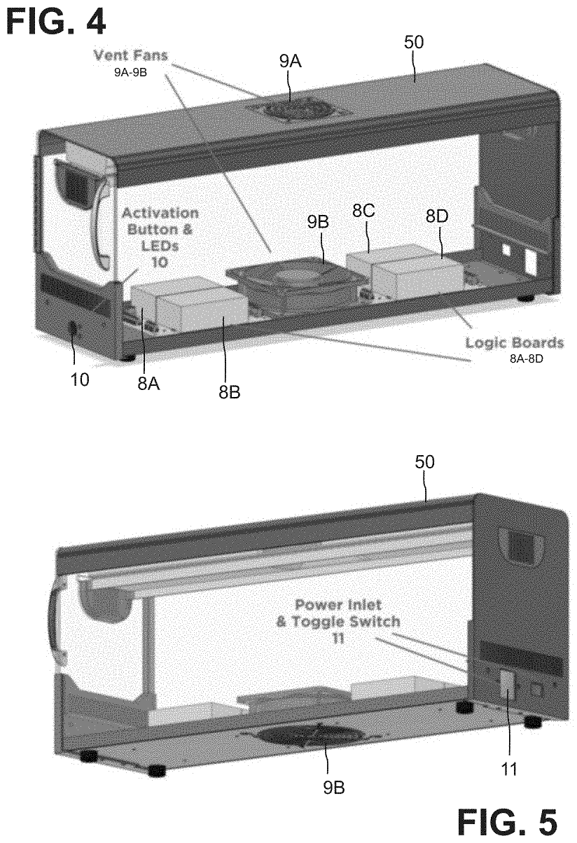

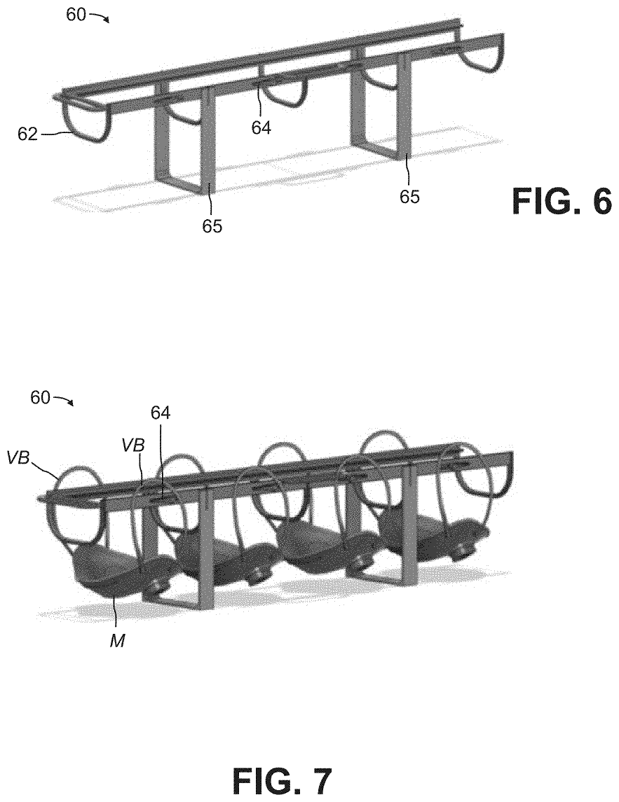

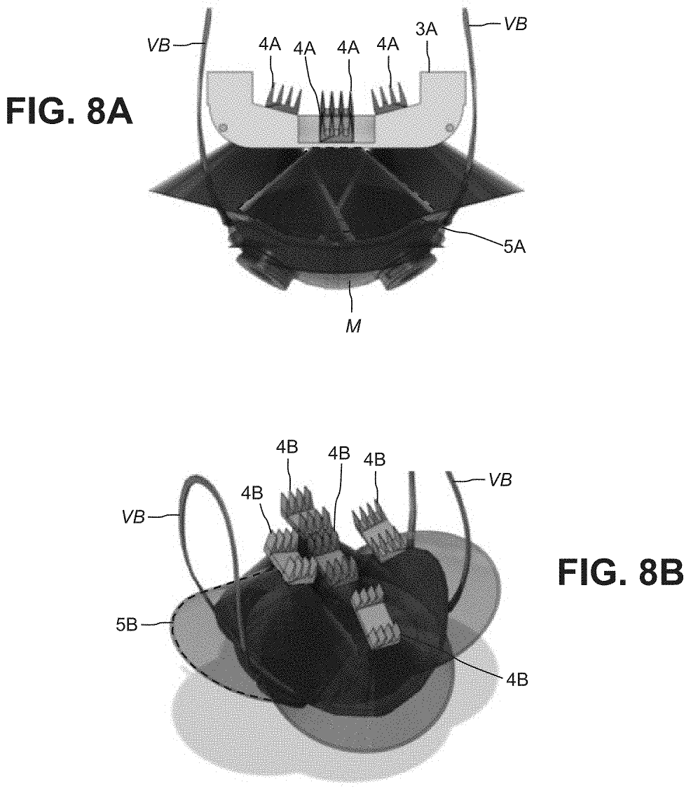

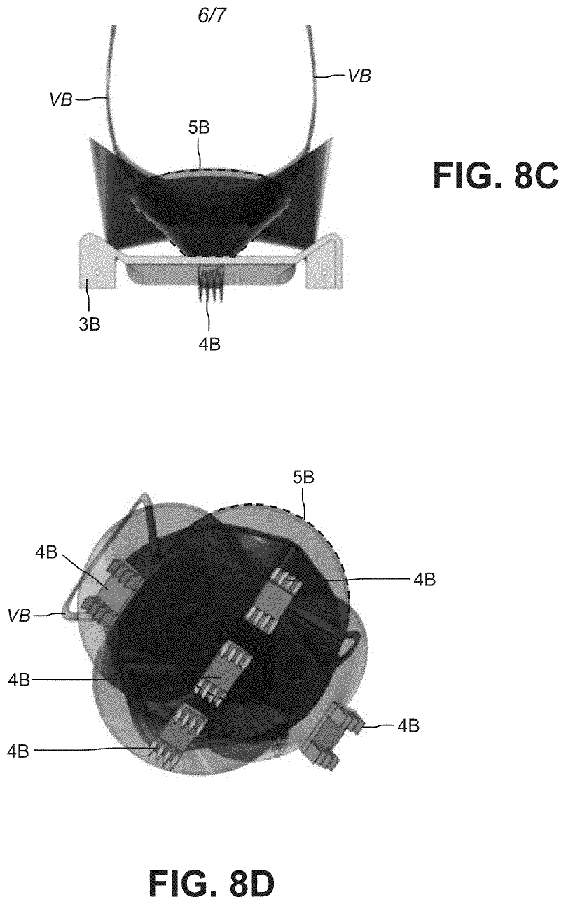

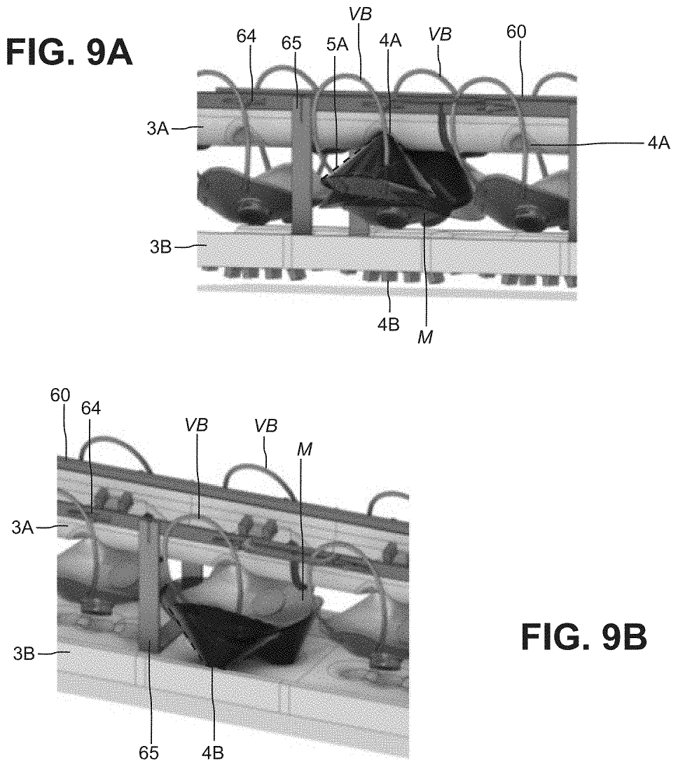

Referring generally to FIGS. 1-9B, the present disclosure provides respirator cleaning apparatus 100 and methods of cleaning a respirator mask M using same.

Respirator cleaning apparatus 100 consistent with the present disclosure employ light— rather than chemical cleansers—to substantially eliminate (e.g., eliminate at least 99%, at least 99.5%, or at least 99.9%) of surface pathogens on a respirator mask M.

Generally, respirator cleaning apparatus 100 consistent with the present disclosure include a housing 50; a superior lamp 4A; an inferior lamp 4B; and a respirator support 60 disposed between the superior lamp 4A and the inferior lamp 4B.

The housing 50 generally defines a chamber in which the respirator mask(s) M are flooded with light. In some embodiments, the housing 50 is formed of a UVC-opaque material, such as glass and/or acrylic. In some embodiments, the housing 50 includes a door 55. The door 55 may be hinged, and may interact with a safety switch (not shown) that automatically and rapidly deactivates the superior lamp 4A and the inferior lamp 4b when the door 55 is opened. In some embodiments, the safety switch prevents the superior lamp 4A and the inferior lamp 4B from illuminating if the door 55 is not fully closed. In some embodiments, the door 55 comprises, consists essentially of, or consists of a UVC-opaque material, such as glass or acrylic.

The respirator support 60 is configured to securely and temporarily position one or more respirator masks M within the chamber in an orientation that enables the superior lamp 4A and the inferior lamp 4B to illuminate all or substantially all of the internal and external surface area of the respirator mask(s) M. In some embodiments, the respirator support 60 is configured to slide into and out of the housing 50. In some embodiments, the respirator support 60 is configured to support a plurality of respirator masks M, such as at least two respirator masks (e.g., 2 respirator masks, 3 respirator masks, 4 respirator masks, 5 respirator masks, 6 respirator masks, 7 respirator masks, 8 respirator masks, 9 respirator masks, 10 respirator masks, or more than 10 respirator masks). In some embodiments, the respirator support 60 includes a reinforcement hoop 62 for associating with each respirator mask M to be housed within the housing 50. In some embodiments, the respirator support 60 includes a stand 65 configured to allow the respirator stand 60 to stand upright on a surface to facilitate convenient loading of respirator masks M to the respirator stand. In some embodiments, the stand 65 enables the respirator masks M to be supported by the respirator support 60 without contacting the surface on which the respirator support 60 rests. In some embodiments, the respirator support 60 includes two visor band clips 64 configured to temporarily retain visor bands VB of each respirator mask M to be housed within the housing 50. In some embodiments, the housing 50 is configured to enable the respirator support 60 to slide into and out of the chamber.

The superior lamp 4A is positioned within the housing 50 above the respirator support 60 to illuminate at least a portion of the respirator mask(s) M within the chamber. In the embodiment specifically illustrated in the accompanying figures, the superior lamp 4A illuminates all or substantially all of the interior surface of the respirator masks M. In other embodiments, the superior lamp 4A is oriented to illuminate all or substantially all of the external surface of the respirator masks M. In still other embodiments, the superior lamp 4A is oriented to illuminate at least a portion of the internal surface of the respirator masks M and at least a portion of the external surface of the respirator masks M. In some embodiments, the superior lamp 4A includes at least two lamps for each respirator mask M housed within the housing 50, such as 2 lamps, 3 lamps, 4 lamps, 5 lamps, 6 lamps, 7 lamps, 8 lamps, 9 lamps, 10 lamps, or more than 10 lamps for each respirator mask M housed within the housing 50. In some embodiments, the superior lamp 4A includes five lamps for each respirator mask housed within the housing. In some embodiments, the superior lamps 4A are housed within a superior lamp tray 3A configured to align a desired number of superior lamps 4A with each respirator mask M within the chamber. In some embodiments, the superior lamp 4A comprises a UVC LED lamp, optionally in thermal communication with a heat sink. In some embodiments, each UVC LED lamp produces a cone of UVC light 5A extending at least about 50 mm from the UVC LED lamp. In some embodiments, the superior lamps 4A are oriented relative to each other such that their respective cones of UVC light 5A overlap with each other to increase the intensity of the produced light in the overlapped regions, and to substantially eliminate all shadowing potential as light contacts every area of the respirator masks M from multiple directions.

The inferior lamp 4B is positioned within the housing 50 below the respirator support 60 to illuminate at least a portion of the respirator mask(s) M within the chamber. In the embodiment specifically illustrated in the accompanying figures, the inferior lamp 4B illuminates all or substantially all of the exterior surface of the respirator masks M. In other embodiments, the inferior lamp 4B is oriented to illuminate all or substantially all of the internal surface of the respirator masks M. In still other embodiments, the inferior lamp 4B is oriented to illuminate at least a portion of the external surface of the respirator masks M and at least a portion of the internal surface of the respirator masks M. In some embodiments, the inferior lamp 4B includes at least two lamps for each respirator mask M housed within the housing, such as 2 lamps, 3 lamps, 4 lamps, 5 lamps, 6 lamps, 7 lamps, 8 lamps, 9 lamps, 10 lamps, or more than 10 lamps for each respirator mask M housed within the housing 50. In some embodiments, the inferior lamp 4B includes five lamps for each respirator mask M housed within the housing 50. In some embodiments, the inferior lamps 4B are housed within an inferior lamp tray 3B configured to align a desired number of inferior lamps 4B with each respirator mask M within the chamber. In some embodiments, the inferior lamp 4B comprises a UVC LED lamp, optionally in thermal communication with a heat sink. In some embodiments, each UVC LED lamp produces a cone of UVC light 5B extending at least about 50 mm from the UVC LED lamp. In some embodiments, the inferior lamps 4B are oriented relative to each other such that their respective cones of UVC light 5B overlap with each other to increase the intensity of the produced light in the overlapped regions, and to substantially eliminate all shadowing potential as light contacts every area of the respirator masks M from multiple directions.

In some embodiments, one or more fans 9A-9B are configured to draw air from around the superior lamps 4A and/or the inferior lamps 4B, and to vent the air out of the housing 50. In some embodiments, the one or more fans 9A-9B are configured to force air into the unit, for example to enhance evaporation of fluid or moisture (e.g., body oils, sweat, etc.) from the surface of the respirator mask(s) M.

One or more logic boards 8A-8D may be in electronic communication with the superior lamps 4A and/or with the inferior lamps 4B, and optionally with the fans 9A-9B, to control operation.

In some embodiments, the present disclosure provides a respirator cleaning apparatus 100 comprising a housing 50; a superior lamp 4A; an inferior lamp 4B; and a respirator support 60 disposed between the superior lamp 4A and the inferior lamp 4B. In some embodiments, the housing 50 includes a door 55. In some embodiments, the respirator support 60 is configured to slide into and out of the housing 50. In some embodiments, the respirator support 60 is configured to support a plurality of respirator masks M. In some embodiments, the superior lamp 4A includes at least two lamps for each respirator mask M housed within the housing. In some embodiments, the superior lamp 4A includes five lamps for each respirator mask M housed within the housing 50. In some embodiments, the inferior lamp 4B includes at least two lamps for each respirator mask M housed within the housing 50. In some embodiments, the inferior lamp 4B includes five lamps for each respirator mask M housed within the housing 50. In some embodiments, the respirator support 60 includes a reinforcement hoop 62 for associating with each respirator mask M to be housed within the housing 50. In some embodiments, the respirator support 60 includes a stand 65. In some embodiments, the respirator support 60 includes two visor band clips 64 configured to temporarily retain visor bands VB of each respirator mask M to be housed within the housing 50. In some embodiments, the superior lamp 4A is configured to illuminate substantially all or all of an interior surface of a respirator mask M housed within the housing 50. In some embodiments, the inferior lamp 4B is configured to illuminate substantially all or all of an exterior surface of a respirator mask M housed within the housing 50. In some embodiments, the superior lamp 4A comprises a UVC LED. In some embodiments, the inferior lamp 4B comprises a UVC LED. In some embodiments, the respirator cleaning apparatus 100 is configured to house four respirator masks within the housing.

In operation, one or more respirator masks M are secured temporarily to the respirator support 60, which is then inserted into the chamber defined by the housing 50. The housing 50 is closed (e.g., the door 55 is closed), and the respirator cleaning apparatus 100 is electrified (e.g., by activating a power switch 11 or other control button). The superior lamps 4A and inferior lamps 4B then illuminate the respirator mask(s) M with cleansing light (e.g., UVC light) until pathogens on the surface of the respirator mask(s) M (hereinafter “surface pathogens”) are eliminated or substantially eliminated (e.g., killed, denatured, or deactivated, as appropriate). In some embodiments, the superior lamps 4A and inferior lamps 4B are illuminated until at least about 99% of surface pathogens, for example at least 99%, at least 99.1%, at least 99.2%, at least 99.3%, at least 99.4%, at least 99.5%, at least 99.6%, at least 99.7%, at least 99.8%, at least 99.9%, at least 99.95%, at least 99.99%, at least 99.999%, or at least 99.9999% of surface pathogens are eliminated from the respirator mask(s) M housed within the chamber. The superior lamps 4A and inferior lamps 4B are then deactivated, and thereafter the respirator support 60 is removed from the chamber and the respirator mask(s) M can be removed from the respirator support 60 for re-use.

In some embodiments, the superior lamps 4A and the inferior lamps 4B are illuminated for not more than about 60 minutes, for example, not more than 60 minutes, not more than 55 minutes, not more than 50 minutes, not more than 45 minutes, not more than 40 minutes, not more than 35 minutes, not more than 30 minutes, not more than 25 minutes, not more than 20 minutes, not more than 15 minutes, not more than 10 minutes, not more than 5 minutes, not more than 4 minutes, not more than 3 minutes, not more than 2 minutes, or not more than 1 minute. In some embodiments, the superior lamps 4A and the inferior lamps 4B are illuminated for not more than about 5 minutes.

In some embodiments, the present disclosure provides a method of cleaning a respirator mask M, the method comprising: providing a respirator cleaning device 100 of as disclosed herein; inserting the respirator mask M into the housing 50; illuminating the respirator mask M with the superior lamp 4A and with the inferior lamp 4B for a period of time sufficient to incapacitate substantially all or all pathogens associated with the respirator mask M; and thereafter removing the respirator mask M from the housing 50. In some embodiments, the period of time is not more than about 5 minutes (e.g., about 5 minutes, about 4 minutes, about 3 minutes, about 2 minutes, or about 1 minute). In some embodiments, the method further comprises applying a hydrophobic or superhydrophobic coating to the respirator mask M before the step of inserting the respirator mask M into the housing 50. In some embodiments, the method does not include applying a chemical cleanser to the respirator mask M. In some embodiments, the step of inserting the respirator mask M into the housing comprises: removing or substantially removing the respirator support 60 from the housing 50; attaching the respirator mask M to the respirator support 60; and reinserting the respirator support 60 into the housing 50.

In the foregoing detailed description, numerous details, examples, and embodiments of the invention are described. However, it will be clear and apparent to one skilled in the art that the technology disclosed herein is not limited to the embodiments set forth and that the technology disclosed herein can be adapted for any of several applications.

### Examples

Example 1. A respirator cleaning apparatus comprising:
a housing;
a superior lamp;
an inferior lamp; and
a respirator support disposed between the superior lamp and the inferior lamp.

Example 2. The respirator cleaning apparatus of Example 1, wherein the housing includes a door.

Example 3. The respirator cleaning apparatus of Example 1 or Example 2, wherein the respirator support is configured to slide into and out of the housing.

Example 4. The respirator cleaning apparatus of any one preceding Example, wherein the respirator support is configured to support a plurality of respirator masks.

Example 5. The respirator cleaning apparatus of any one preceding Example, wherein the superior lamp includes at least two lamps for each respirator mask housed within the housing.

Example 6. The respirator cleaning apparatus of Example 5, wherein the superior lamp includes five lamps for each respirator mask housed within the housing.

Example 7. The respirator cleaning apparatus of any one preceding Example, wherein the inferior lamp includes at least two lamps for each respirator mask housed within the housing.

Example 8. The respirator cleaning apparatus of Example 7, wherein the inferior lamp includes five lamps for each respirator mask housed within the housing.

Example 9. The respirator cleaning apparatus of any one preceding Example, wherein the respirator support includes a reinforcement hoop for associating with each respirator mask to be housed within the housing.

Example 10. The respirator cleaning apparatus of any one preceding Example, wherein the respirator support includes a stand.

Example 11. The respirator cleaning apparatus of any one preceding Example, wherein the respirator support includes two visor band clips configured to temporarily retain visor bands of each respirator to be housed within the housing.

Example 12. The respirator cleaning apparatus of any one preceding Example, wherein the superior lamp is configured to illuminate substantially all or all of an interior surface of a respirator mask housed within the housing.

Example 13. The respirator cleaning apparatus of any one preceding Example, wherein the inferior lamp is configured to illuminate substantially all or all of an exterior surface of a respirator mask housed within the housing.

Example 14. The respirator cleaning apparatus of any one preceding Example, wherein the superior lamp comprises a UVC LED.

Example 15. The respirator cleaning apparatus of any one preceding Example, wherein the inferior lamp comprises a UVC LED.

Example 16. The respirator cleaning apparatus of any one preceding Example, wherein the respirator cleaning apparatus is configured to house four respirator masks within the housing.

Example 17. A method of cleaning a respirator mask, the method comprising:
providing a respirator cleaning device of any one preceding claim;
inserting the respirator mask into the housing;
illuminating the respirator mask with the superior lamp and with the inferior lamp for a period of time sufficient to incapacitate substantially all or all pathogens associated with the respirator mask; and
thereafter removing the respirator mask from the housing.

Example 18. The method of Example 17, wherein the period of time is not more than about 5 minutes.

Example 19. The method of Example 17 or Example 18 further comprising applying a hydrophobic or superhydrophobic coating to the respirator mask before the step of inserting the respirator mask into the housing.

Example 20. The method of any one of Examples 17-19, wherein the method does not include applying a chemical cleanser to the respirator mask.

Example 21. The method of any one of Examples 17-20, wherein the step of inserting the respirator mask into the housing comprises:
removing or substantially removing the respirator support from the housing;
attaching the respirator mask to the respirator support; and
reinserting the respirator support into the housing.

What is claimed is:

1. A respirator cleaning apparatus comprising:
a housing;
a superior lamp;
an inferior lamp; and
a respirator support disposed between the superior lamp and the inferior lamp.

2. The respirator cleaning apparatus of claim 1, wherein the housing includes a door.

3. The respirator cleaning apparatus of claim 1, wherein the respirator support is configured to slide into and out of the housing.

4. The respirator cleaning apparatus of claim 1, wherein the respirator support is configured to support a plurality of respirator masks.

5. The respirator cleaning apparatus of claim 1, wherein the superior lamp includes at least two lamps for each respirator mask housed within the housing.

6. The respirator cleaning apparatus of claim 5, wherein the superior lamp includes five lamps for each respirator mask housed within the housing.

7. The respirator cleaning apparatus of claim 1, wherein the inferior lamp includes at least two lamps for each respirator mask housed within the housing.

8. The respirator cleaning apparatus of claim 7, wherein the inferior lamp includes five lamps for each respirator mask housed within the housing.

9. The respirator cleaning apparatus of claim 1, wherein the respirator support includes a reinforcement hoop for associating with each respirator mask to be housed within the housing.

10. The respirator cleaning apparatus of claim 1, wherein the respirator support includes a stand.

11. The respirator cleaning apparatus of claim 1, wherein the respirator support includes two visor band clips configured to temporarily retain visor bands of each respirator to be housed within the housing.

12. The respirator cleaning apparatus of claim 1, wherein the superior lamp is configured to illuminate substantially all or all of an interior surface of a respirator mask housed within the housing.

13. The respirator cleaning apparatus of claim 1, wherein the inferior lamp is configured to illuminate substantially all or all of an exterior surface of a respirator mask housed within the housing.

14. The respirator cleaning apparatus of claim 1, wherein the superior lamp comprises a UVC LED.

15. The respirator cleaning apparatus of claim 1, wherein the inferior lamp comprises a UVC LED.

16. The respirator cleaning apparatus of claim 1, wherein the respirator cleaning apparatus is configured to house four respirator masks within the housing.

17. A method of cleaning a respirator mask, the method comprising:
providing a respirator cleaning device of claim 1;
inserting the respirator mask into the housing;
illuminating the respirator mask with the superior lamp and with the inferior lamp for a period of time sufficient to incapacitate substantially all or all pathogens associated with the respirator mask; and
thereafter removing the respirator mask from the housing.

18. The method of claim 17, wherein the period of time is not more than about 5 minutes.

19. The method of claim 17 further comprising applying a hydrophobic or superhydrophobic coating to the respirator mask before the step of inserting the respirator mask into the housing.

20. The method of claim 17, wherein the method does not include applying a chemical cleanser to the respirator mask.

21. The method of claim 17, wherein the step of inserting the respirator mask into the housing comprises:
removing or substantially removing the respirator support from the housing;
attaching the respirator mask to the respirator support; and
reinserting the respirator support into the housing.
